(19) United States
(12) Patent Application Publication
(10) Pub. No.: US 2024/0428415 A1
(43) Pub. Date: Dec. 26, 2024

(54) SYSTEM AND METHOD FOR MONITORING TREATMENT THROUGH PET-CT COMBINATION IMAGING

(71) Applicants: SHEBA IMPACT LTD., Ramat-Gan (IL); A.Y.Y.T. - TECHNOLOGICAL APPLICATIONS AND DATA UPDATE LTD., Holon (IL)

(72) Inventors: Tima DAVIDSON, Holon (IL); Johnatan NISSAN, Ramat-Gan (IL); Amit MELAMED, Holon (IL); Moriel GINDI, Holon (IL); Dror MALKA, Netanya (IL)

(73) Assignees: SHEBA IMPACT LTD., Ramat-Gan (IL); A.Y.Y.T. - TECHNOLOGICAL APPLICATIONS AND DATA UPDATE LTD., Holon (IL)

(21) Appl. No.: 18/684,119

(22) PCT Filed: Aug. 15, 2022
(86) PCT No.: PCT/IL2022/050883

§ 371 Date: Feb. 15, 2024

Related U.S. Application Data
(60) Provisional application No. 63/234,414, filed on Aug. 18, 2021.

(51) Int. Cl. G06T 7/00; G06T 7/11; G06T 7/174; G06T 7/32

(52) U.S. Cl. CPC G06T 7/0016; G06T 7/11; G06T 7/174; G06T 7/32; G06T 2207/10081; G06T 2207/10104; G06T 2207/30004

(57) ABSTRACT

A system for monitoring treatment of a patient includes data input and output utilities, memory, and a data processor, and communicates with an image data provider to receive therefrom image data indicative of combined PET-CT scan images including at least one first and at least one second pre-treatment full body scan image of a patient. The data processor includes an identifier utility that processes the image data to identify matching first and second 2D regions in, respectively, first 2D slices forming the first scan image and second 2D slices forming the second scan image, and locate at least one pair of corresponding first and second 3D regions in the scan images; and an analyzer that analyzes each pair of the first and second 3D regions and determine a change in at least one parameter in the first and second 3D regions, and generate output data indicative of said change.

### TECHNOLOGICAL FIELD

The present invention is generally in the field of image processing techniques used in medical applications, and particularly relates to image processing of a combined imaging technique of Positron Emission Tomography (PET) and computed tomography (CT).

### BACKGROUND

Combined PET and CT modality, commonly referred to as PET-CT, is used to obtain a single superimposed scan of both imaging techniques. Nowadays, the PET-CT modality is an essential imaging tool for assessing patients with various medical diagnoses, particularly in the field of oncology.

CT scan is based on X-ray beams technology and aimed to evaluate anatomical features of the patient's body based on the density of tissues The density scale in CT is measured by Hounsfield units (HUs), which are dimensionless units (obtained from a linear transformation of the measured attenuation coefficients) commonly used to express density in a standardized and convenient form.

PET scan is an imaging modality assessing the metabolic activity of the patient's tissues. The technique involves injecting radioactive material (nowadays, the most common is 18F-fluorodeoxyglucose (FDG)) into the patient's blood circulation, which then accumulates in metabolically active tissues in the patient's body. This radioactive material's accumulation can be measured by the PET modality and introduced as a 3D image of the body. Since cancer tissue often has a higher metabolic rate than normal tissue, a PET scan can differentiate between these tissues. The scale that assesses this accumulation of contrast material in the tissues is measured by Maximum Standardized Uptake Value (SUVₘₐₓ), a simple image-based measurement widely used in clinical practice. SUVₘₐₓ scale is a semiquantitative measurement and is calculated as a dimensionless ratio of the metabolic activity per unit volume in a region of interest (ROI) to the whole body's metabolic activity per unit volume. Metabolic tumor volume (MTV) is the tumor biological target volume measured by PET-CT. Another important parameter is metabolic tumor volume (MTV), which represents the metabolically active volume of the tumor, which is commonly measured by a fixed percentage threshold of SUVₘₐₓ.

The use of PET-CT involves integrating both PET metabolic findings and the anatomical information provided by the CT component. Thus, PET-CT is an important tool to diagnose malignancy and assess staging of cancer. In addition, PET-CT is also a vital tool for planning treatment and evaluating its effectiveness on follow-up scans, mainly among oncological patients The evaluation of the post-treatment follow-up scan is made by assessing the changes between the pre- and post-treatment parameters of both cancer and normal tissues, such as the metabolic changes of the lesions (decreasing, increasing, or even appearing), by using SUVₘₐₓ and MTV by PET, and tumor size and HU levels by CT. Nowadays, doctors are evaluating these changes manually and based on a large number of medical scans.

### General Description

There is a need in the art for a fully or at least almost automated system for radiologists to assist them in identifying changes between follow-up PET-CT scans. More specifically, there is a need for such a system capable of determining and presenting to the radiologist the instant sizes, levels of HU, SUVₘₐₓ and MTV changes for possible faster and more accurate diagnostics.

The present invention provides a novel system and method for evaluation of the changes between two following PET-CT scans. The technique developed by the inventors draws the radiologist's attention to the suspicious regions change, mainly pre- and post-treatment, and assess automatically the changes of various parameters of the lesion\region of interest between the scans.

According to one broad aspect of the invention, there is provided a system for monitoring body treatment of a patient, the system comprising data input and output utilities, memory, and a data processor, and being configured for data communication with an image data provider to receive, from the image data provider, image data indicative of combined PET-CT scan images including at least one first pre-treatment full body scan image of a patient and at least one second post-treatment full body scan image of the patient. The data processor is configured and operable (pre-programmed) to process the image data to identify matching 2D regions in first 2D slices forming the first pre-treatment full body scan image and second 2D slices forming the second post-treatment full body scan image, and locate at least one pair of corresponding/complementary 3D regions in the first and second full body scan images, and analyse each of said at least one pair of the complementary 3D regions and determine a change in at least one parameter of interest in paired pre-treatment and post-treatment 3D regions, and generate output data indicative of said change.

In the description below, the terms “pre-treatment” and “post-treatment” are interchangeably used with the terms “before treatment” and “after treatment”, respectively.

The data processor is configured and operable to represent each of a first set of 2D slices forming the first full body scan image (before treatment) and a second set of 2D slices forming the second full body scan image (after treatment) by respective first 2D logical images and second 2D logical images. Such first and second 2D logical images correspond to, respectively, first Coronal-first Sagittal images and second Coronal-second Sagittal images.

Thus, combined PET-CT full body scan image data obtained from each patient before and after the treatment is represented by Coronal before, Sagittal before, Coronal after and Sagittal after 2D images. These 2D images are processed and analysed to identify matching 2D regions in the before- and after-treatment images, and to locate corresponding matching 3D regions in the PET-CT full body scan image data before and after the treatment.

The technique of the present invention thus provides fully automatic analysis of PET-CT scan image data and presentation of reliable results to radiologist with respect to any change in suspicious regions appearing after the treatment.

In some embodiments, said data processor includes a transformation utility; a matching utility; and an analyser. The transformation utility is configured to transform 3D data indicative of the first and second full body scan images into respective first and second pairs of 2D logical images. As mentioned above, the 2D logical images of the first pair correspond to a first Coronal image and a first Sagittal image of the patient's body before the treatment, and the 2D logical images of the second pair correspond to a second Coronal image and a second Sagittal image of the patient's body after said treatment.

It should be noted that in some embodiments, the data processor may be configured and operable to perform some pre-processing of the raw data by dividing the set of data to PET and CT and normalizing by SUV level; and then filtering the PET images to obtain suspicious regions.

The matching utility is configured to define matching 2D regions in the first and second pairs of the 2D logical images, wherein each pair of the matching 2D regions is defined by first 2D regions in the first Coronal and first Sagittal images matching with respective second 2D regions in the second Coronal and second Sagittal images. As will be described below, the matching utility may be configured and operable to perform cross-correlation methods, preferably followed by validation process.

The analyser is configured to analyse each pair of the first and second matching 2D regions over the first and second 3D scan images and locate a corresponding pair of matching first and second 3D regions in the first and second full body scan images. The first 3D region in the first full body scan image corresponds to the first 2D regions in the first Coronal and first Sagittal images, and the second 3D region in the second full body scan image corresponds to the second 2D regions in the second Coronal and Sagittal images, thereby determining data about the at least one pair of the 3D regions to be analysed to identify the change before and after the treatment.

In some embodiments, the data about the at least one pair of the 3D regions to be analysed includes, for each pair of the 3D regions, its associated 2D regions before treatment (in the first Coronal and Sagittal images) and 2D regions after the treatment (in the second Coronal and Sagittal images).

In some embodiments, the results of the above data analysis provide a solid matching matrix between the 3D suspicious region before the treatment to its corresponding 3D suspicious region after said treatment. This enables to evaluate the changes in one or more parameters of the matching regions. Such parameter(s) includes at least one of the following: volume, activity level (SUV) and HU levels. Also, the results of this data analysis can show new regions that emerged and old regions that disappeared.

In some embodiments, the data processor comprises a pre-processor configured to pre-process raw data indicative of said first and second full body scan images to define said 3D data to be processed by the data transformation utility. The pre-processor may comprise: a normalization utility configured to normalize data corresponding to each of the first and second full body scan images to a predetermined scale (e.g. corresponding to SUV) thereby obtaining respective first and second normalized full body scan images; and a filtering utility configured to apply spatial and thresholding filtering to each of said first and second full body normalized scan images and provide corresponding first and second sets of filtered 2D PET slices forming said first and second full body scan images, respectively.

The data transformation utility is preferably further configured and operable to apply a dimension reduction procedure to the filtered 2D PET-CT slices to optimize the 2D logical images.

Also, the data transformation utility is preferably configured to perform registration of each of the 2D logical images of the first pair in X- and Y axes to correspond to shifts of the patient's body between scans during imaging. To this end, the data transformation utility may be configured to perform segmentation of each of the 2D logical images to define each individual region in the 2D logical image, and determine parameters of each of the individual regions; and perform matching of at least some of said parameters of each individual region of the first 2D logical image before registration with parameters of the region in the first 2D logical image after registration.

In some embodiments, the match finding utility is configured to represent each of the 2D regions in 3D space and in 2D space by performing 2D Cross-Correlation, to determine a distance, R, by which the first pre-treatment image is to be moved for matching the second post-treatment image, for each of the Coronal and Sagittal 2D images.

The above may be performed as follows:
applying first Cross-Correlations to each 2D region of the first pre-treatment Coronal 2D image with respect to all 2D regions in the second post-treatment Coronal image, and to each 2D region of the first pre-treatment Sagittal 2D image with respect to all 2D regions in the second post-treatment Sagittal image, thereby obtaining a first Coronal matrix, CZ^{(BT)}×CZ^{(AT)}, with a size CZ^{(BT)} of pre-treatment Coronal regions on a size CZ^{(AT)} of post-treatment Coronal regions, and a first Sagittal matrix, SZ^{(BT)}×SZ^{(AT)} with a size SZ^{(BT)} of pre-treatment Sagittal regions on a size SZ^{(AT)} of post-treatment Sagittal regions, each cell in the matrix representing the distance R;
applying a second Cross-Correlation in determined limits to the regions of pre-treatment and post-treatment Coronal and Sagittal images regions and obtaining, for each of the Coronal and Sagittal images, a first matrix of a size of number of pre-treatment regions on a size of post-treatment regions, and a second matrix of the size of number of after-treatment regions on the number of pre-treatment regions, content of the first and matrixes being the distance R;
thereby obtaining six matrixes comprised of three matrixes for the Coronal image and three matrixes for the Sagittal image.

The match finding utility may be further configured to optimize a matching process by carrying out the following: applying revoking to each of said six matrixes to revoke one or more illogical matches, being a match of the region in the pre-treatment image to several regions in a corresponding post-treatment image; and merging resulting six matrixes into 2D Coronal-Merge matrix and 2D Sagittal-Merge matrix.

Preferably, the match finding utility is configured to verify validity of the Coronal-Merge and Sagittal-Merge matrixes to regions of the post-treatment Coronal and Sagittal images that have not passed through the registration, and thereby obtain two 2D matrixes including relations between the regions without registration, for the pre-treatment and post-treatment Coronal and Sagittal images.

According to another broad aspect of the invention, there is provided a method for monitoring treatment of a patient, the method comprising:
providing image data indicative of combined PET-CT scan images including at least one first pre-treatment full body scan image of a patient and at least one second post-treatment full body scan image of the patient,
processing the image data to identify matching first and second 2D regions in, respectively, first 2D slices forming the first pre-treatment full body scan image and second 2D slices forming the second post-treatment full body scan image, locate at least one pair of corresponding/complementary first and second 3D regions in the first and second full body scan images, respectively, and analyse each of said at least one pair of the first and second 3D regions to determine a change in at least one parameter of interest in the first and second 3D regions, and generate output data indicative of said change.

The technique of the invention provides for automatic presentation of user interfaces enabling a user (physician, radiologist) to activate various functionalities of the above-described algorithm and obtain the results presented in the interface. Such functionalities include one or more of the following:

The algorithm can receive a full PET-CT scan as an input and produce an output of full-body 3D, and 2D images of the PET-CT scan with automatic markings of all the suspicious findings/lesions. This interface allows the physician to focus on the most important findings in the image without distractions from the less important findings and tough save critical time. In addition, the algorithm can receive a full PET-CT scan as input and produce an output of full-body 3D and 2D images of the PET-CT scan with a “heat map” that presents the changes from the previous scan. This interface eliminates the need to manually choose a specific finding in order to examine its difference from the previous scan.

This user interface can help the physician to analyse and write the scan interpretation more quickly. Moreover, the system can generate an automated report of all the findings in the scans and their difference from the previous test. This report could, for instance, be recorded in the patient medical documentation or be used for research.

### BRIEF DESCRIPTION OF THE DRAWINGS

In order to understand the invention and to see how it may be carried out in practice, embodiments will now be described, by way of non-limiting example only, with reference to the accompanying drawings. Features shown in the drawings are meant to be illustrative of only some embodiments of the invention, unless otherwise implicitly indicated. In the drawings like reference numerals are used to indicate corresponding parts, and in which:

FIG. 1 is a block diagram of the main functional utilities of the control system of the present invention;

FIG. 2 more specifically illustrates an exemplary configuration and operation of a data processor in the system of the invention;

FIG. 3 exemplifies a segment of 3D image and associated 2D Coronal and 2D Sagittal views thereof, obtained according to the principles of the present invention;

FIG. 4A shows an exemplary flow diagram of the operation of the data processor in the system of the invention;

FIG. 4B exemplifies a flow diagram of the operation of a match finding utility of the control system of the invention for defining a 3D region from collection of PET-CT scan images before treatment to its corresponding 3D region from the collection after treatment;

FIGS. 5a-f exemplify the results of the filtering technique suitable to be used in the present invention, as a part of the pre-processing procedure, wherein FIGS. 5a,b illustrate pre- and after treatment 2D slices before filtering, FIGS. 5c,d the corresponding pre- and after treatment slices after spatial filtering; and FIGS. 5e,f illustrate respective pre- and after treatment slices after the “Double Thresh” filtering;

FIG. 6 shows a 3D module of the suspicious regions after all the filtering and pre-processing has been done to each 2D slice;

FIGS. 7a-b illustrate the principles of dimension reduction processing suitable to be used in the present invention based on presenting 2D data as the projection of 3D data from two different perspectives, Coronal view (FIG. 7a) and Sagittal view (FIG. 7b) to obtain 2D logical images;

FIGS. 8a,b exemplify the results of a registration procedure suitable to be used in the present invention, wherein FIG. 8a shows Coronal views (before and after treatment) before registration, and FIG. 8b shows the Coronal views (before and after treatment) after registration;

FIGS. 9a-f exemplify Coronal before-treatment image (FIG. 9a) and regions resulting from the segmentation procedure of such image (FIGS. 9b-f);

FIG. 10 illustrates an example for a matching matrix for Coronal before-treatment and after-treatment thresholding processing;

FIGS. 11a-c exemplify the process of limiting a giving region suitable to be used in the present invention for the purposes of matching presentation between the regions;

FIGS. 12a-f exemplify the principles of revoking procedure suitable to be used in the present invention;

FIG. 13 is a flow diagram of the exemplary revoking procedure;

FIG. 14 exemplifies images before registration (right side) and after registration (left side);

FIG. 15 is a flow diagram exemplifying a procedure of matching between the original image and image after registration;

FIGS. 16a-c exemplify, respectively, a Coronal image after registration, a Coronal image before registration, and a Coronal image after treatment, i.e. non-registered; and

FIG. 17 is a flow diagram exemplifying a process of correcting a match matrix for the case that several regions forming the 3D collection before treatment are united to one 3D region after treatment.

It is noted that the embodiments exemplified in the figures are not intended to be in scale and are in diagram form to facilitate ease of understanding and description.

### DETAILED DESCRIPTION OF EMBODIMENTS

One or more specific and/or alternative embodiments of the present disclosure will be described below with reference to the drawings, which are to be considered in all aspects as illustrative only and not restrictive in any manner. It shall be apparent to one skilled in the art that these embodiments may be practiced without such specific details. In an effort to provide a concise description of these embodiments, not all features or details of an actual implementation are described at length in the specification. Elements illustrated in the drawings are not necessarily to scale, or in correct proportional relationships, which are not critical.

For case of reference, common reference numerals or series of numerals will be used throughout the figures when referring to the same or functionally similar features common to the figures.

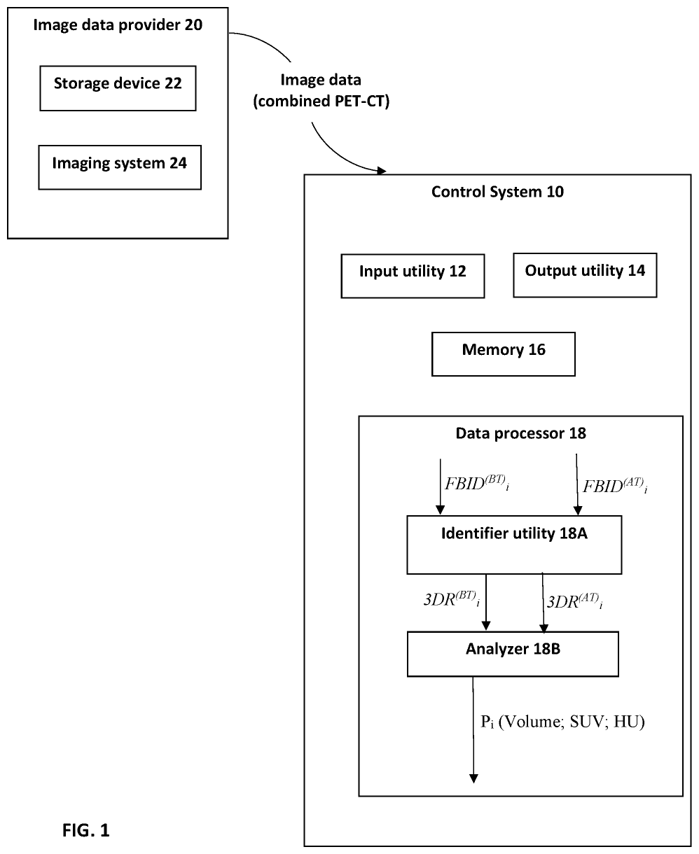

Referring to FIG. 1, there is shown, by way of a block diagram, a control system 10 configured and operable according to the invention for monitoring treatment of a patient by performing analysis of combined PCT-CT image data obtained before and after said treatment. The system 10 is configured as a computer system including inter alia such functional parts as a data input utility 12, data output utility 14, memory 16, and data processor 18.

The system 10 is configured for data communication with an image data provider 20, which may be constituted by an external storage device 22 where such image data is stored (being obtained from a combined PET-CT imaging system), or by the storage utility of the imaging system 24 itself. The system 10 may communicate with an external image data provider 20 using any known data communication technique and protocols. Alternatively, the control system may communicate with an “internal” system, i.e., the system 10 may be integral with the imaging system 24. In some other embodiments, functional utilities of the control system may be distributed between an internal controller of the imaging system and a remote data processing system. The system 10 may thus also include appropriate communication utility. The configuration of such communication utility, using any known suitable technology, is known per se and therefore need not be specifically described.

The imaging system 24 may also be of any known suitable configuration including a combined FDG-PET/CT scanner. Such a combined PCT-CT scanner typically includes the CT and PET components (detector arrays) mounted on a single rotating support and the data acquired from two separate types of detectors is then aligned/registered in combined images. The construction and operation of the combined PET-CT imaging system is known per se and do not for part of the present invention, and therefore need not be specifically described, except not note that for the purposes of the present invention, the image data to be processed and analysed is indicative of combined PCT-CT scan images collected from the whole body of a patient (at times referred to hereinbelow as “whole body” or “full body” image data) before and after a certain treatment.

Thus, the control system 10 receives raw data including the combined PCT-CT full body scan image data (FBID) from the image data provider 20. This image data is typically 3D data being indicative of one or more 3D images, each formed by a set of 2D slices.

According to the invention, for each i-th patient, the image data that is to be provided and is to be processed and analysed by the control system 10 includes data indicative of at least one pair of first and second images, being the first pre-treatment full body scan image FBID^{(BT)} (acquired before the treatment) and the second post-treatment full body scan image FBID^{(AT)}, (acquired after said treatment).

The data processor 18 is configured to apply to this image data, FBID^{(BT)}ᵢ and FBID^{(AT)}_{I} an image processing technique configured according to the invention to automatically identify changes between regions (suspicious regions) in the before- and after-treatment full body images to evaluate the effect of the treatment.

The data processor includes: an identifier utility 18A which processes the first and second full body scan images FBID^{(BT)}ᵢ and FBID^{(AT)}_{I} to locate matching pair(s) of first and second 3D regions 3DR^{(BT)}ᵢ-3DR^{(AT)}ᵢ in these images respectively; and an analyser 18B which analyses each such pair of matching 3D regions 3DR^{(BT)}ᵢ-3DR^{(AT)}, to determine a change between them in at least one parameter of interest, and generate output data indicative of the identified changes. The parameters of interest typically include one or more of volume, activity level (SUV) and HU levels.

The data processor 18 is thus configured to process 3D scan image data of the combined PET-CT scans obtained before and after the treatment to determine a change in the patient's medical condition (e.g., tumour condition). This enables accurate monitoring of the disease development.

The identifier utility 18A is configured and operable to carry out the following: identify, in first 2D slices forming the first pre-treatment full body scan image FBID^{(BT)}ᵢ and second 2D slices forming the second post-treatment full body scan image FBID^{(AT)}ᵢ, matching first and second 2D regions, respectively; and locate at least one pair of corresponding/complementary first and second 3D regions in the first and second full body scan images, respectively.

To this end, the data processor 18A operates to present each of the input 3D full body scan images by a set of 2D body images of the front and side views (Coronal and Sagittal views), and process such 2D images to find matching 2D regions before and after the treatment, and then define corresponding matching 3D regions in 3D data indicative of the input 3D image scans. This allows to identify suspicious regions and compare their appearance in the before- and after-treatment combined PET-CT image data.

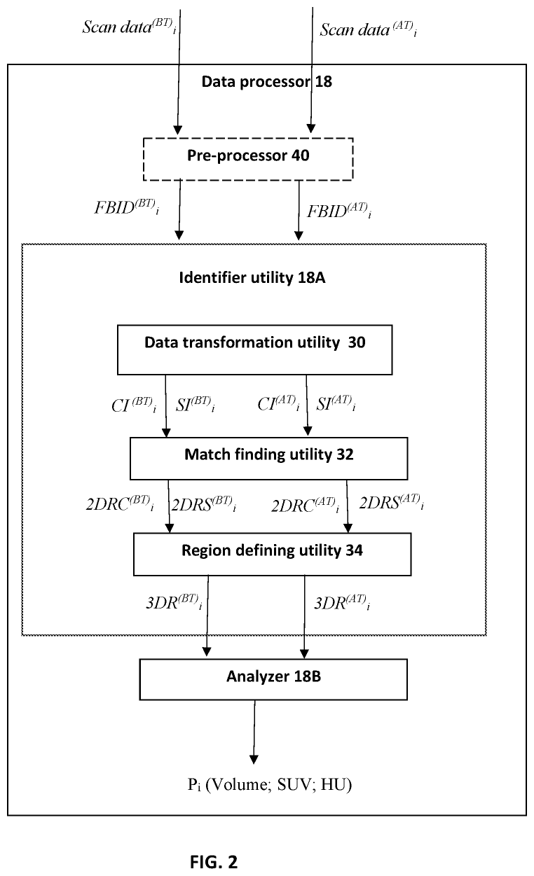

Reference is now made to FIG. 2 more specifically exemplifying configuration and operation of the data processor 18, in particular the identifier utility 18A. The identifier utility 18A includes a data transformation utility 30, a match finding utility 32, and a region defining utility 34.

The data transformation utility 30 receives 3D data indicative of the first pre-treatment full body scan image data FBID^{(BT)}, of the i-th patient and the second post-treatment full body scan image data FBID^{(AT)}ᵢ, and operates to transform such 3D data into respective first and second pairs of 2D logical images. The data corresponding to pre-treatment 3D scan image FBID^{(BT)}_{I} (a set of slices forming such image) is transformed into a first 2D logical Coronal image CI^{(BT)}_{I} and a first 2D logical Sagittal image SI^{(BT)}ᵢ of the patient's body; and data corresponding to the post-treatment 3D scan image FBID^{(AT)}_{I} (a set of slices forming such image) is transformed into a second 2D logical Coronal image CI^{(AT)}ᵢ and a second 2D logical Sagittal image SI^{(AT)}ᵢ of the patient's body. Thus, the input 3D scan image data is now transformed such that the slices of the pre- and post-treatment 3D scans are represented by two pairs of 2D logical images, i.e. four 2D logical images: 2D Coronal pre-treatment image CI^{(BT)}ᵢ, 2D Sagittal pre-treatment image SI^{(BT)}ᵢ, 2D Coronal post-treatment image CI^{(AT)}ᵢ, and 2D Sagittal post-treatment image SI^{(BT)}ᵢ:
FBID^{(BT)}_{I}→CI^{(BT)}ᵢ& SI^{(BT)}ᵢ
FBID^{(AT)}_{I}→CI^{(AT)}ᵢ& SI^{(AT)}ᵢ

The so-obtained data indicative of the 2D logical images is processed by the match finding utility 32. The latter is configured to define, in these 2D logical images, one or more pairs of matching 2D regions. The pair of matching 2D regions is defined by first (pre-treatment) 2D regions in the first Coronal and Sagittal images, 2DRC^{(BT)}ᵢ and 2DRS^{(BT)}, which match (within a certain pre-defined degree) with respective second (post-treatment) 2D regions in the second Coronal and Sagittal images 2DRC^{(AT)}ᵢ and 2DRS(AT)ᵢ.

Thus, the transformation and match finding utilities provide four 2D regions (Coronal-before 2DRC^{(BT)}ᵢ and Sagittal-before 2DRS^{(BT)}ᵢ regions and Coronal-after 2DRC^{(AT)}_{I} and Sagittal-after 2DRS^{(AT)}ᵢ regions) in the respective Coronal-before and Sagittal-after 2D logical images satisfying a matching condition:

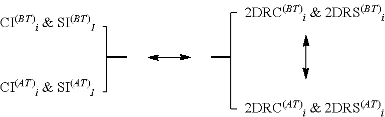

The matching procedure will be exemplified more specifically further below.

The region defining utility 34 is configured to determine a solid matching matrix between the one or more 3D regions before treatment (pre-treatment) to corresponding one or more 3D regions post-treatment (after treatment). This enables to identify the changes in one or more parameters of the matching regions. Also, this shows new region(s) that emerged, as well as shows old regions that disappeared.

More specifically, the region defining utility 34 receives the above 2D matching regions data and analyse each pair of the matching 2D regions over 3D data indicative of the first and second full-body scan images FBID^{(BT)}ᵢ and FBID^{(AT)}ᵢ to define and locate a corresponding pair of matching first and second 3D regions therein, 3DR^{(BT)}ᵢ and 3DR^{(AT)}_{I}, respectively. Here, the first 3D region 3DR^{(BT)}ᵢ corresponds to the pre-treatment 2D regions in the Coronal-before and Sagittal-before, and the second 3D region 3DR^{(AT)}ᵢ corresponds to the post-treatment 2D regions in the Coronal-after and Sagittal-after images:

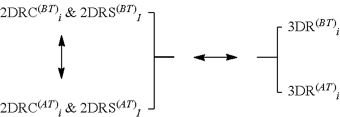

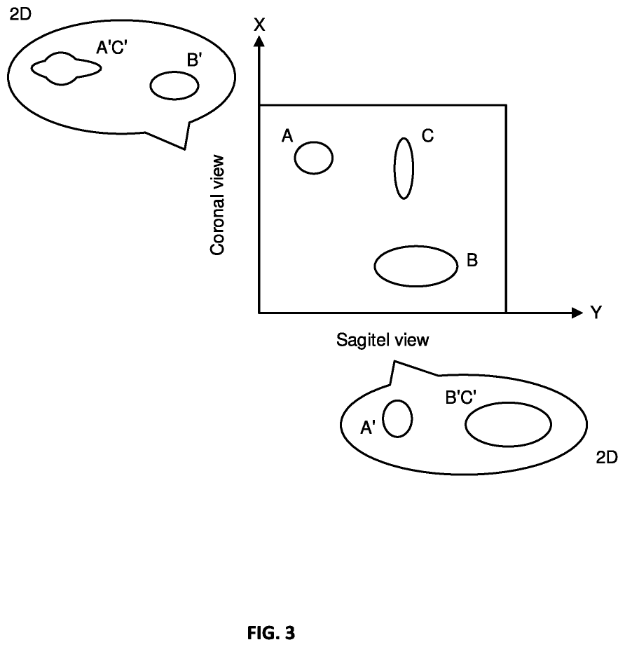

Thus, each 3D region in each of the full-body scan image (before and after-treatment scan images) is associated/assigned with its corresponding 2D regions of the Coronal and Sagittal images. This is exemplified in FIG. 3, showing a segment of 3D image and associated 2D Coronal and 2D Sagittal views thereof.

In the specific not-limiting example of FIG. 3, some 3D regions overlap each other in the 2D images: there are overlapping 3D regions in both of the 2D Coronal and 2D Sagittal views (regions A′C and B′C′) and their 3D segment (A,B,C). Hence, the overlapping regions should preferably be classified. This will be described more specifically further below.

Such data about the one or more pairs of matching 3D regions in the pre- and post-treatment PET-CT scans is provided, to be analysed to identify suspicious region and determine the change for each pair of such suspicious regions before and after the treatment. A suspicious area/region is typically defined as an area/region whose corresponding SUV level is higher than a chosen thresh value, given by a physician.

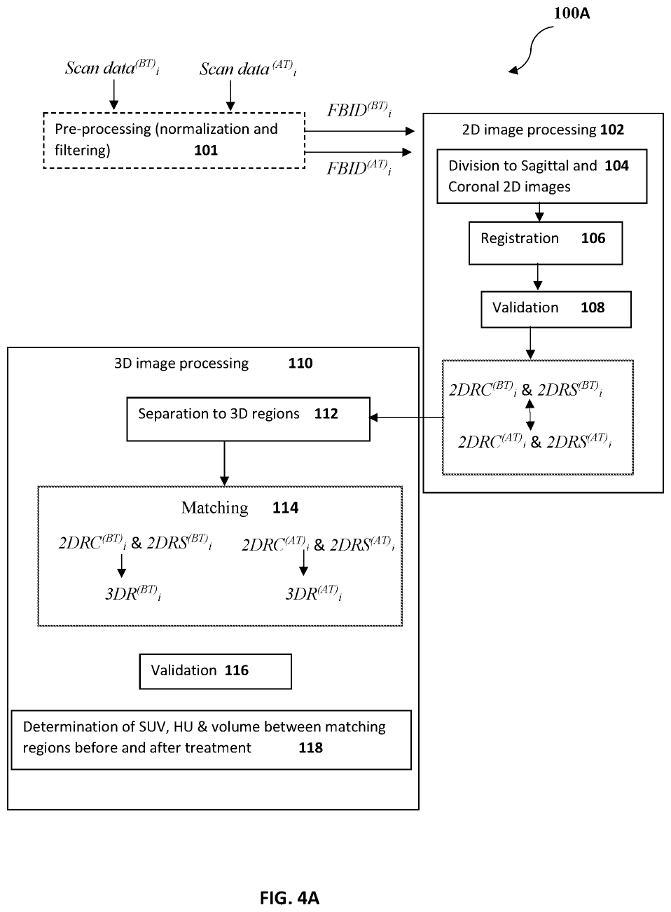

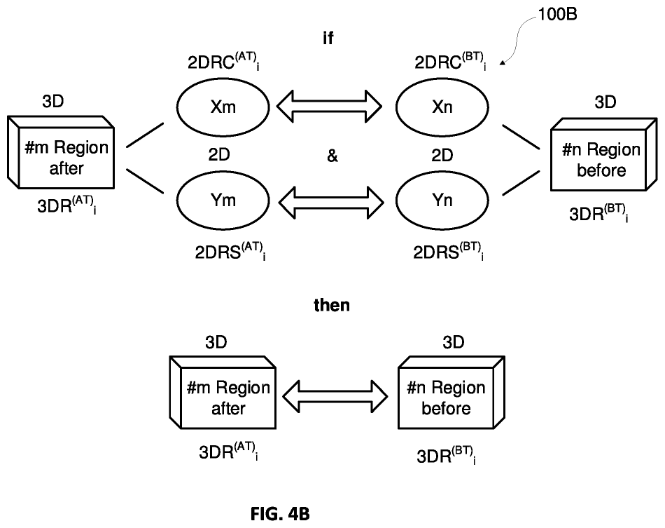

FIG. 4A shows an exemplary flow diagram 100A of the operation of the processor 18. As shown in the figure, the data processor 18 applies 2D image processing stage 102 to the set of 2D slices of the pre-treatment full body scan image data FBID^{(BT)}, and the set of 2D slices of the post-treatment full body scan image data FBID^{(AT)}_{I} of the patient. This 2D processing stage includes division of this image data into Sagittal and Coronal 2D images (step 104), and registration between these Sagittal and Coronal 2D images (step 106) preferably followed by validation procedure (step 108), as will be described more specifically further below.

The 2D image processing stage results the pair of 2D regions in the pre-treatment Coronal and Sagittal images, 2DRC^{(BT)}ᵢ and 2D) RS^{(BT)}_{I} which match 2D regions in the post-treatment Coronal and Sagittal images 2DR^{(AT)}ᵢ and 2DRS^{(AT)}ᵢ. This data under 3D image processing stage 110. This stage includes separation to 3D regions (step 112), and matching procedure (step 114) aimed at defining and locating a corresponding pair of matching first (pre-treatment) and second (post-treatment) 3D regions, 3DR^{(BT)}ᵢ and 3DR^{(AT)}_{I}, respectively. This can be followed by validation process (step 116), and determination of such parameters as SUV, HU and volume between matching regions before and after treatment (step 118).

The output of the processor may be in the form of a printed matrix that shows the correlation between 3D regions before the treatment and their matching 3D region(s) after treatment. The system may also save data about the SUV level change of each set of corresponding's regions before and after treatment. The system provides a fully automated user interface presenting the changes of HU, SUV and Volume differences of each lesion.

FIG. 4B shows an exemplary flow diagram 100B illustrating, in a self-explanatory manner, the principles of the above-described technique performed by the match finding utility 32 for defining a 3D region 3DR^{(BT)}ᵢ from the pre-treatment collection to its corresponding 3D region 3DR^{(AT)}ᵢ from the post-treatment collection. The arrows represent matching between given regions. Only if a pair of 3D regions (in the pre- and post-treatment image data) is defined by matching Coronal and Sagittal 2D regions 2DRC^{(BT)}ᵢ-2DRC^{(AT)}ᵢ and 2DRS^{(BT)}ᵢ-2DRS^{(AT)}_{I}, then and only then, the giving 3D regions are set to be paired (i.e., matching regions).

Turning back to FIGS. 2 and 4A, there is illustrated that the data processor 18 optionally but preferably also includes a pre-processor 40. Pre-processing (step 101 in FIG. 4A) is applied to the input combined PET-CT full body scan data Scan data^{(BT)}ᵢ and Scan data^{(AT)}ᵢ collected by the combined PET-CT imaging system before and after treatment, respectively. The pre-processor 40 is configured to apply to such input data normalization and filtering procedures. This is associated with the following:

The input full body scan image data is typically raw data, i.e. a package/unit including multiple types of data. For example, in the experiments conducted by the inventors (which will be described more specifically further below), the PET-CT data was provided in the form of a unit of 16 data types. Thus, such input image data needs to be normalized to a familiar scale (such as SUV), and preferably also further filtered to facilitate transformation into 2D logical images and finding matching regions therein.

As indicated above, the 3D scan image is formed by a set of 2D slices. The normalization and filtering processing is successively applied to each of the slices of each of the pre- and post-treatment 3D scan images.

The SUV normalization is a factor of the patient weight, active isotope dose and the scan time. The SUV can be calculated by using the following equation

$$SUV = \frac{Activity Concentrationin R O I}{( injected Dose / Body Weight )} \qquad ( 1 )$$

wherein Activity Concentrationin ROI is the activity for a given volume of the region of interest (ROI) at the image acquisition time; the Injected Dose is the dose at the injection time;

and Body Weight is the weight of the patient in kg.

The proper filtering, applied to each of the normalized 2D slices, includes a spatial filtering, which may be 3D Gaussian Filter followed by Sharpen Filter. Also, due to the nature of PET-CT image noise (Poision noise), a Wiener Filter may be applied.

Then, the so-obtained “filtered” 2D slices undergo thresholding filtering (based on at least one predefined thresh). In this connection, it should be noted that in order to enable further extraction of the suspicious areas/regions, the use of a single threshold might not be sufficient to obtain the desirable and eliminate the undesirable regions, and the use of two thresholds (“Double Thresh”) is more suitable for this task.

The first main thresh value Thresh₁ is selected to pass (enable selection of) pixels P₁ having pixel value PV₁ greater than or equal to said thresh, i.e. PV₁≥Thresh₁. The second thresh value Thresh₂ is selected to pass (enable selection of) pixels P₂ whose pixel value PV₂ is greater than said second thresh, i.e., PV₂>Thresh₂, and which satisfy a condition that at least one of neighbouring pixels is the P/pixel, i.e. the at least one neighbouring pixel of pixel P₂ has passed the first, main, thresh Thresh₁.

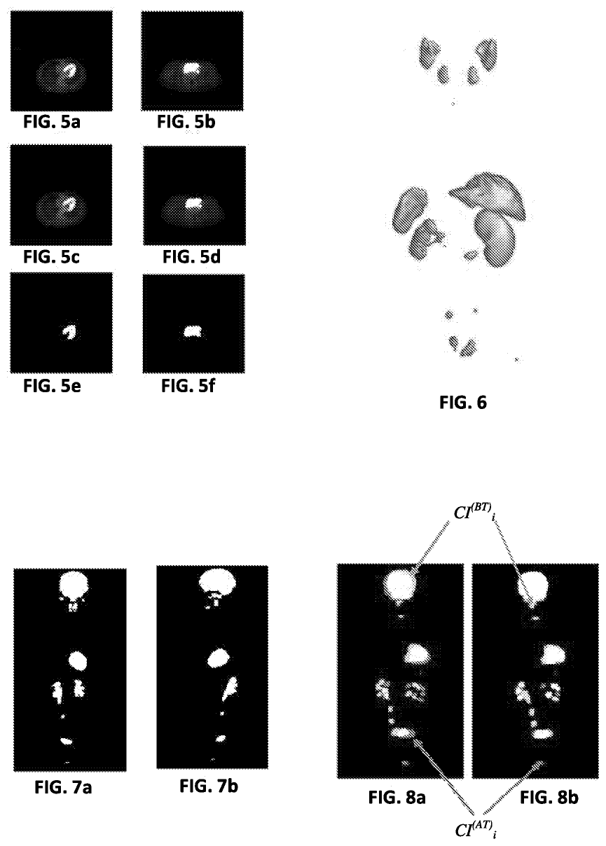

FIGS. 5a-f exemplify comparison between pre- and post-treatment slices before and after filtering. More specifically, FIGS. 5a,c,e show the pre-treatment slices before filtering, after spatial filtering, and after “Double Thresh” processing, respectively; and FIGS. 5b,d,f) show the same stages for the post-treatment slices.

After applying the above-described spatial filtering and thresholding (“Double Thresh”) processing to the slices of the FBID^{(BT)}ᵢ and FBID⁽⁴⁷⁾ᵢ scans images, the resulted slices (exemplified in FIGS. 5e,f) may be converted to a binary format (not SUV scale) and the resulted slices undergo the transformation procedure, which is generally described above.

FIG. 6 shows a 3D module of the suspicious regions after all the filtering and pre-processing has been done to each 2D slice. After obtaining the pre-processed 2D slices, the creation of the coronal and sagittal 2D images is performed.

Preferably, the transformation utility 30 is also configured to apply a dimension reduction procedure to the filtered 2D slices/images of the FBID^{(BT)}ᵢ and FBID^{(AT)}ᵢ scans images to optimize the 2D images to be more “logical” for further transformation and analysis. To this end, each 2D slice of the pre-treatment scan image FBID^{(BT)}, and each 2D slice of the post-treatment scan image FBID^{(AT)}ᵢ are converted into respective 2D logical images. Since each scan (before treatment/after treatment) is constructed from a number of slices, each scan can be seen as a 3D Data Matrix. Thus, each segment/area is a 3D region inside a 3D space. In order to simplify the further task of matching 3D regions between the scans, the dimension reduction processing is performed, meaning that 2D data/image is observed as the projection of the 3D data from two different perspectives, the first Coronal (frontal view) and the second Sagittal (lateral view).

This is exemplified in FIGS. 7a-b showing a 2D logical image of the 3D data (obtained in the experiments) from Coronal view (FIG. 7a) and Sagittal view (FIG. 7b).

The transformation utility 30 is preferably further configured to perform image registration in order to optimize the region defining task. Such registration requirement is associated with the body shift along X and Y axes due to different alignment of patient between the scans. The registration technique used in the present invention is based on the general principles of 2D Intensity Based Registration and utilizes two different techniques of geometric transformation, the XY translation and a rigid transformation including translation and rotation (to reject the possibility of major body shift). Eventually, the selected technique is the one which provides the greatest congruence (highest degree of similarity) between the related before- or after-treatment 2D images (one for Coronal view and one for Sagittal view).

It should be emphasized that the registration procedure is applied only on the 2D Coronal/Sagittal images before treatment.

FIGS. 8a,b illustrate 2D Coronal views (taken from a specific patient in the experiments conducted by the inventors) before registration (FIG. 8a) and after registration (FIG. 8b). In this example, each figure illustrates regions forming the pre-treatment image CT^{(BT)}ᵢ (green) and regions forming the post-treatment image CI^{(AT)}_{I} (purple). The white regions in the figures correspond to congruent areas/regions between the scans. It can be noticed that corresponding regions are aligned after the registration.

In order to enable identification of each region in the 2D images for matching purposes, the transformation utility 30 also preferably operates to perform segmentation of the 2D image to properly define each independent (individual) region/segment in the 2D image. To this end, the individual region is defined by a collection of pixels of value 1 surrounded by pixels with a value 0. For example, Matlab's Region Props Analysis can be used for extracting those individual segments/regions and determining their parameters, such as XY location, size, etc.

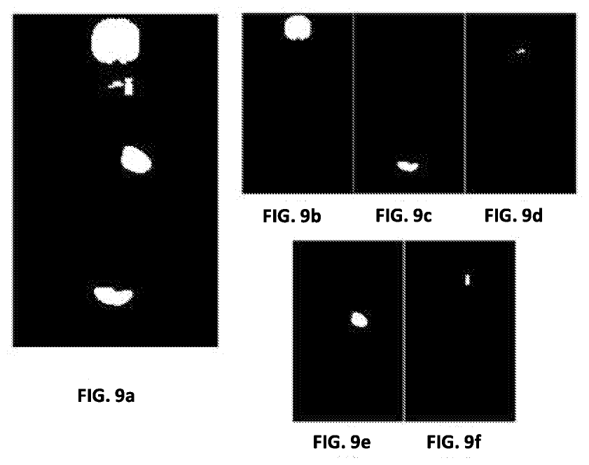

This is exemplified in FIGS. 9a-f, showing an example for Coronal pre-treatment image (FIG. 9a) and regions resulting from the segmentation procedure of such image (FIGS. 9b-f). For data preservation, such as position and area (size), each region/segment of the 2D images (pre-treatment images) before registration is matched with the images after registration.

As described above, the match finding utility 32 processes the first (before-treatment) and second (after-treatment) pairs of the 2D logical images, CI^{(BT)}ᵢ-SI^{(BT)}ᵢ and CI^{(AT)}ᵢ-ST^{(AT)}ᵢ and defines one or more pairs of matching 2D regions therein. The match finding utility 32 may utilize the representation of the 2D regions (and their related spatial data such as position and size) in 3D space and also in 2D space (before and after treatment) as described above. For this purpose, two 2D Cross-Correlation techniques can be used. The Cross-Correlation is a measure of similarity of two series, i.e. two images, and is given by:

$$C ( k , l ) = {\sum }_{m = 0}^{M - 1} {\sum }_{n = 0}^{N - 1} X ( m , n ) \cdot H ( m - k , n - l ) \qquad ( 2 )$$

$$- ( P - 1 ) \leq k \leq M - 1$$

$$- ( P - 1 ) \leq k \leq M - 1$$

wherein X is an (M×N) size matrix, H is a (P×Q) size matrix, and C is the Cross-Correlation result in [(P+M−1)×(Q+N−1)] size matrix.

The result of the Cross-Correlation provides a matrix in which the maxima (pixel) indicates the position of the best Correlation. By this, the distance, R, can be extracted which is a distance by which the first image is to be moved for matching the second image.

$$X = {X}_{maximum} - {X}_{c e n t e r} \qquad ( 3 )$$

$$Y = {Y}_{maximum} - {Y}_{c e n t e r}$$

$$R = \sqrt{{X}^{2} + {Y}^{2}}$$

wherein (Xₘₐₓᵢₘᵤₘ, Yₘₐₓᵢₘᵤₘ) are the coordinates of a point Tₘₐₓ of the Cross-Correlation maximum; (X_{center}, Y_{center}) are the coordinates of a point T_{center} of the Cross-Correlation center, and R is the distance between these two points.

As mentioned above, in order to define the matching 2D regions, two techniques using Cross-Correlation can be used. In the first technique, each 2D region from the pre-treatment collection undergoes Cross-Correlation with all 2D regions in the post-treatment collection and the distance R for each Cross-Correlation is determined (eq.3). This is performed for Coronal separately and Sagittal separately: each 2D region in the Coronal before-treatment images CI^{(BT)}ᵢ is Cross-correlated with 2D regions in the Coronal after-treatment images; and each 2D region in the Sagittal before-treatment image SI^{(BT)}ᵢ surpasses Cross-Correlation with all 2D regions in the Sagittal after-treatment image CI^{(AT)}ᵢ).

The calculation result yields a matrix with the size Z^{(BT)} of the regions before-treatment on the size Z^{(AT)} of the regions after-treatment (one for Coronal and one for Sagittal), i.e. two matrixes: matrix CZ^{(BT)}×CZ^{(AT)} and matrix SZ^{(BT)}×SZ^{(AT)}. Each cell in the matrix represents the distance R between each two regions. After filling the matrix, a certain threshold is chosen to eliminate obvious unpassable matching cases (distance wise).

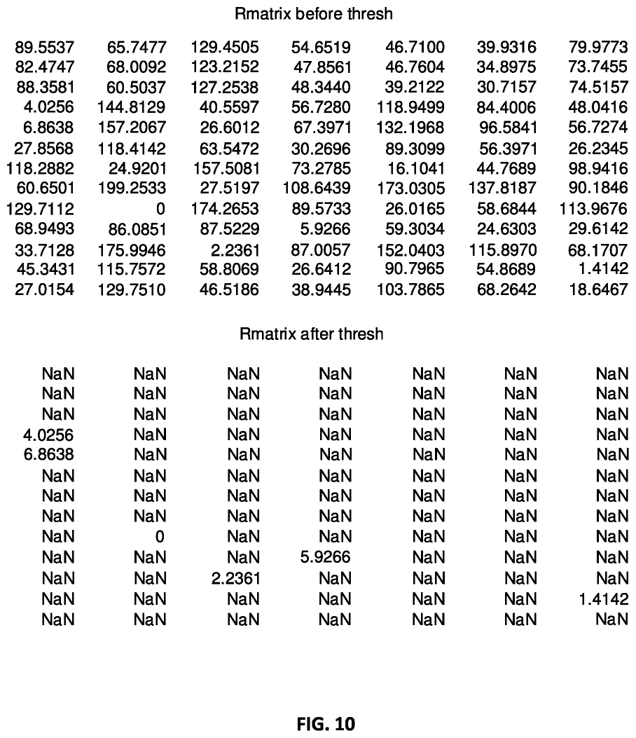

FIG. 10 illustrates an example for a matching matrix for Coronal before-treatment and after-treatment thresholding. The elimination of vast distances is noticeable.

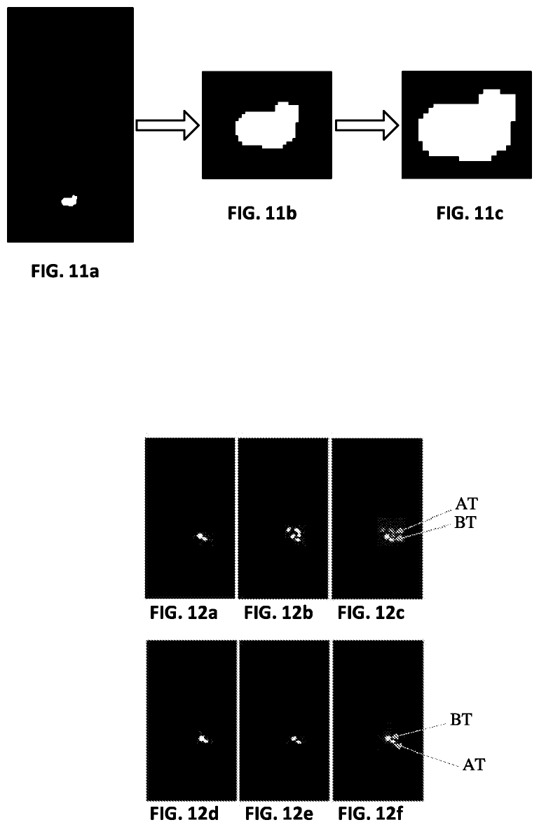

The second technique is based on Cross-Correlation in determined limits. This is carried out in two stages: regions before-treatment are compared with regions after-treatment and vice versa. The matching presentation can be done by limiting each region before treatment for its own edges into a square, and padding with zeros its edges by a given size. This is illustrated in FIGS. 11a-c showing the process of limiting a giving region (FIG. 11a) to that of FIG. 11b) and zero padding (FIG. 11c).

After the padding, every 2D region in the after-treatment image is approached and cropped to the limit of the region that is being tested. More specifically, according to this technique, after the padding has been done, each 2D region after treatment is cropped to the same size of the cropped 2D before-treatment region and Cross-Correlation is performed. After that, the next before-treatment region is cropped and padded as well, and once again the cutting procedure is repeated for all of the 2D after-treatment regions.

Then, a 2D Cross-Correlation between the two temporary images is performed and the distance R is extracted (i.e., the distance by which the first image is to be moved for matching the second image). As mentioned above, the second stage is the opposite of the above-described one, i.e. the cropped and padding procedures are first applied to the after-treatment regions, and then each 2D after-treatment region undergoes Cross-Correlation with all before-treatment cropped regions to its size.

Then, regions of the after-treatment image and the regions of the before-treatment image are tested (analysed). This two-stage technique can be used to solve a problem that one region matches to several regions from the complementary scan, as will be described further below.

Lastly, two matrixes are defined: the first is a matrix of the size of number of regions before treatment on the size of regions after treatment, and the second is a matrix of the size of number of regions after treatment on the number of regions before treatment. The content of these two matrixes is the distance R. It should be noted that each two matrixes are for each view (Coronal/Sagittal). Hence, in the second technique, four matrixes of matching are obtained.

After calculating the above matrixes from the two Cross-Correlation techniques, six matrixes at this stage, three matrixes for Coronal view and three for Sagittal view (for each view, one matrix from the first technique and two matrixes from the second technique), the matching procedure can be further optimized by revoking matches that appeared to be illogical. To this end, it is assumed that that a before-treatment region can be matched to several after-treatment regions in case the volume (number of pixels)/size of after-treatment regions is smaller or equal to the volume/size of the before-treatment region.

Such matches can be revoked by carrying out the following: Any region from the collection before treatment (2D before-treatment image/slices) is approached and examined to determine whether it matches to more than one region from the collection after treatment (2D before-treatment image/slices). The area of the single before-treatment region and the area of several after-treatment regions is calculated, and these areas are analysed: Upon determining that the total area of the several after-treatment regions is greater than the area of the single before-treatment region, the region from the after-treatment collection, which has the greatest distance, R, is rejected from the before-treatment region.

Then, a new area of all the after-treatment regions is calculated without the revoked region, and the above steps are repeated until the area of the single before-treatment region is greater/equal to the areas of the after-treatment regions, or until there is only one region left from the after-treatment collection.

FIGS. 12a-f exemplify the above-described revoking procedure. Here, FIGS. 12a,d show a single region from the before-treatment collection. This is a region from the pre-treatment 2D Coronal image that had been matched to several regions in the post-treatment 2D Coronal image. FIG. 12b shows its matching regions obtained by the calculation from the after-treatment collection (this is the image of all the regions together from the after-treatment image). FIG. 12c shows the overlay of regions of FIGS. 12a,b (i.e. regions before and after treatment). FIG. 12e shows the remaining areas from the calculated areas after the revoking procedure. As can be seen, only two regions from the after-treatment image are assigned to the before-treatment region. FIG. 12f shows the overlay of region of FIGS. 12a,d (before treatment) and regions of FIG. 12e (after treatment).

The revoking procedure is applied on each of the six matching matrixes, obtained in the Cross-Correlation techniques. Then, merging of the six matching matrixes is performed, three for Coronal image and three for Sagittal image, into the two final 2D matrixes, Coronal-Merge and Sagittal-Merge matrixes, that present matching between the regions for each view. After obtaining the “merged” matrixes, the revoking procedure is performed again to provide greater accuracy and to eliminate or at least significantly reduce the possibility that a mistake occurred in one of the six matrixes.

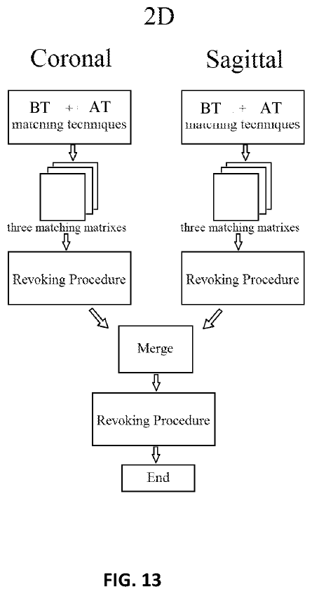

The above process is illustrated in a self-explanatory manner in a flow diagram of FIG. 13.

As described above with regards to the registration procedure, this is applied to the before-treatment images, i.e. to the 2D logical coronal image and 2D logical sagittal image. The registration algorithm changes the images (some regions may be influenced and merged to other regions because of the registration procedure); hence, for the volume changes, the original data (without registration) is to be used. The final matching results are applied on the regions that did not go throw the registration. After this, the final two matrixes for 2D regions can be obtained.

The following is the exemplary technique of how the pre-registration data is used.

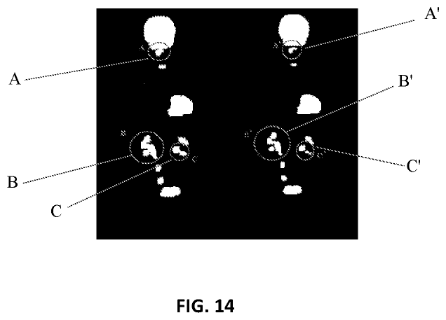

In order to determine the changes in volume, radius and activity levels, HU and SUV, the post-treatment scans are to be compared to the original (non-registered) pre-treatment scans. To this end, matching is to be performed between the registration results for the pre-treatment images and the original pre-treatment images. FIG. 14 exemplifies images before registration (right side) and after registration (left side), showing regions A, B, C in the after-registration image and their matching regions A′, B′ and C′ in the before-registration image.

Generally, in order to match the regions before registration (BR) to the regions after registration (AR), the following is performed:

Data indicative of the original image before registration is used in order to extract therefrom each of the independent/individual regions (as described above with reference to FIGS. 9a-f, these are regions defined by a collection of pixels of value 1 surrounded by pixels with a value 0). Then, each region of the AR image is processed/treated individually, and the tested region is subtracted (e.g., by applying the selected transformation similar to that described above). If the answer is 0 (the resulted region has no positive pixels), then a match is found and said region in the before-registration image corresponds to that region in the after-registration image (matching region).

The accepted result is a matrix having a size of a number of regions in the before-registration image on a size of a number of regions in the after-registration image, which includes a match between the respective regions.

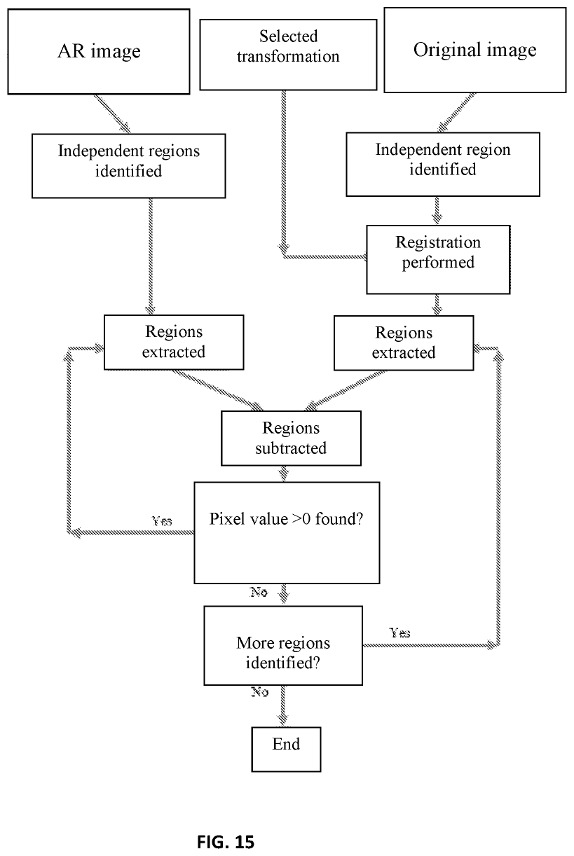

The above-described procedure of matching between the original image and image after registration is illustrated in a self-explanatory manner in a flow diagram of FIG. 15.

It should be noted that in the description above, the obtained matrixes Coronal-Merge and Sagittal-Merge describe relation between the coronal and sagittal pre-treatment after-registration images and the coronal and sagittal post-treatment images. Hence, the original images (i.e. without registration) can now be combined/united.

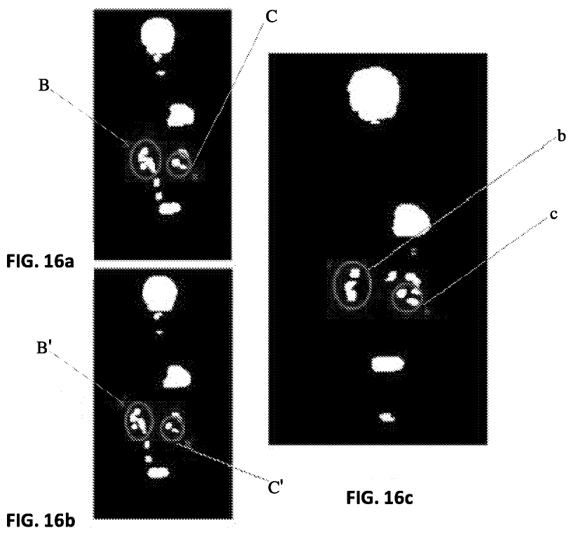

Indeed, as described above, the matrix of the size of number of regions in the before-registration image on the size of number of regions in the after-registration image has been obtained. This data can be combined with the Coronal-Merge and Sagittal-Merge matrixes resulting from the Cross-Correlation analysis and relating to the pre- and post-treatment images. In this connection, reference is made to FIGS. 16a-c exemplifying an image after registration (FIG. 16a), image before registration (FIG. 16b) and after-treatment Coronal image, i.e. non-registered (FIG. 16c). It should be noted that the regions that have been matched to the after-treatment images (as described above) are to be correct/correspond to the before-treatment images. All the results are to be taken from the before-treatment image because this image contains the original data. It can be seen that the registration indeed combines a number of independent regions in the original image: regions B′ and C′ are combined in the after-registration image.

Thus, the regions that have been combined in the registration are to be analyzed to determine whether the Coronal-Merge and Sagittal-Merge matrixes are still correct/valid for such regions. In this connection, the Coronal-Merge matrix provides that regions B and b (FIGS. 16a,c) being, respectively, in the before-treatment with no registration image and after-treatment region, satisfy a matching condition; and the matrix describing the regions before and after registration (as described above) provides that region B and region B′ (before treatment with no registration) are matching regions (FIGS. 16a,b).

Let us now verify whether regions B′ and b are matching, based on the transitiveness condition (whenever A=B and B=C then A=C). Regarding the regions b and B′, the verification can be implemented as follows: apply the selected registration to each sub-region within region B′; subtract each sub-region of region b from region B′; determine whether the number of negative pixels (pixel value “0”) in the resulted image (after subtraction) is smaller than the number of pixels in region b. If this is the case then the resulted image is included in a segment of region B′ from which it has been subtracted (i.e., matching condition); otherwise there is no match.

As a result of the above processing, two 2D matrixes are obtained including relations between the regions without any registration, for coronal and sagittal images before and after treatment.

Turning back to FIG. 2, the results of the 2D regions matching procedure exemplified above are then used by the region defining utility 34 which analyses each of the one or more pairs of the matching 2D regions over the 3D data indicative of the first (before-treatment) and second (after-treatment) full-body scan images in order to define and locate a corresponding pair of matching first and second 3D regions. This can be performed by manipulating the 2D matching matrixes calculated as described above via detection of the connectivity between the 2D regions and their corresponding 3D regions, and then by combining the 2D final results with their corresponding 3D regions.

In order to achieve comparability between the 2D regions and their corresponding 3D regions, the 3D regions can be approached and each one is individualized similar to that done with respect to the 2D regions as described above. After that, Coronal and Sagittal projections of each 3D regions are produced, as described above with respect to the 2D regions. The difference in this procedure is that some 3D regions overlap each other in the 2D images, and thus the overlapping regions are to be classified.

Turning back to FIG. 3, each 3D region is defined by two 2D regions from Coronal view and Sagittal view, one from each view. As shown in the figure, there are overlapping 3D regions in both 2D Coronal and 2D Sagittal view (A′C′, B′C′) and their 3D regions (A,B,C). The regions A′C′ (Coronal view) and B′C′ (Sagittal view) are a combination of regions A, C and B,C, respectably. Moreover, region C is defined as the union of regions A′C′ and B′C′. Hence, any matching for the 3D region C occurs between regions A′C′ and B′C′ to their corresponding 2D region from the final matching matrix for the 2D view. Thus, two pairs of matching 2D regions are to be located, one pair from Coronal before-treatment to Coronal after-treatment and one from Sagittal before-treatment to Sagittal after-treatment, and determine which 3D region before-treatment and after-treatment is defined by the Coronal and Sagittal 2D regions.

Subsequently, all of the 3D pairs are obtained that have been matched to a matrix structure that shows the connection from each 3D region before treatment to its corresponding 3D regions after treatment. The revoking procedure can be applied on the obtained matrix, which procedure is generally similar to that described above for the 2D case, but utilizing comparison of volumes of the regions, and not areas, for 3D regions.

Let us consider the case that several regions forming the 3D collection before treatment are united to one 3D region after treatment. Couple of regions are matched to one region if and only if for each region before treatment, the multiplying results of the register coronal and sagittal projections with the corresponding coronal and sagittal projections of the one region after treatment are non-zero. Then, the tested region is considered matching the one region after treatment. Otherwise, the connection is to be revoked. The final connection matrix for 3D region is remained.

More specifically, it might be the case that there are 3D regions that appear to be overlapping with 2D regions, i.e. 2D scan shows an individual region, while there are actually two or more 3D regions. As a result, we can obtain a match between 2D regions, which actually do not correspond to a match between corresponding 3D regions. This is for example the case when two or more regions in pre-treatment 3D scan become combined into a single region.

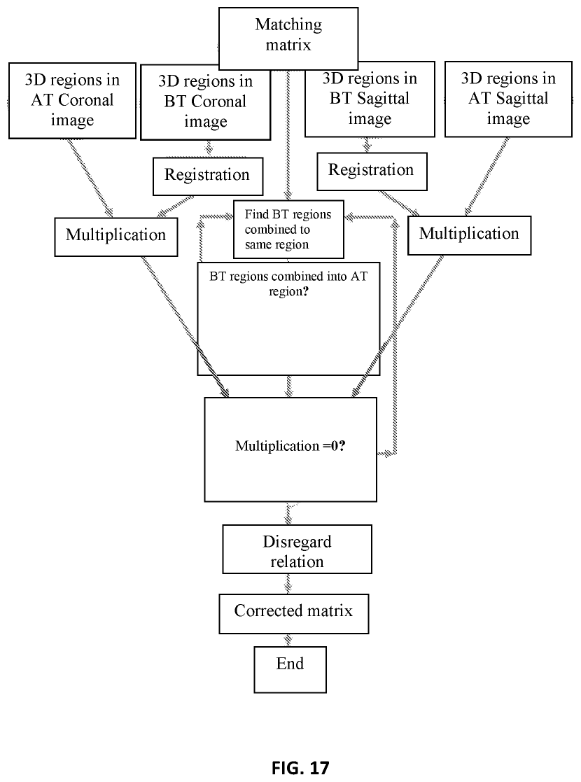

To this end, the analysis similar to that described above with reference to FIGS. 3 and 4 is performed. This is exemplified in a flow diagram of FIG. 17.

Each region in the pre-treatment and after-treatment 3D scans is analysed as being presented by coronal and sagittal 2D images (four images). The regions of the pre-treatment 2D image undergo registration as described above, and regions that have undergone registration are multiplied by regions that have not been registered, i.e., regions of the pre-treatment coronal and sagittal images that have undergone registration, with the regions after treatment. Then, the matching 3D matrixes are used to identify matching regions before and after treatment, and determine the multiplication result for the matching regions. If there is any 0 result (i.e. there is no agreement in one of the views), then the respective region in the pre-treatment 3D scan does not correspond to 3D region in the post-treatment scan. At this stage, the final matrix is obtained. This matrix shows connections between 3D regions in the before-treatment images to 3D regions in the after-treatment images. Also, this matrix shows new regions that were added (in the after-treatment image) and regions that disappeared (as compared to the before-treatment image).

In the experiments conducted by the inventor, all the image data was extracted from the PET-CT scans of nine different patients, before and after medical treatment (two scans for each patient). All the scans were full body PET-CT, which included a series of whole body CT, PET and Fusion. The patients were both male and female in all ages, and were under medical follow-up. All the data was in a Dicom format that includes all the slices that construct the body (3D) and a Metadata file on each slice. The information in the Metadata includes valid information on the scan and the patient, such as Slice Location, Patient Weight, Modality (CT/PET), etc.

Imaging data obtained for five patients was used for training and testing the operation of the control system of the invention by Leave-One-Out Cross Validation (LOOCV), and imaging data for the other four patients was used solely for testing the system.

In these experiments, the FDG-PET/CT scanner commercially available from Philips Gemini GXL, Philips Medical Systems, Cleveland OH, USA was used. Such scanner includes a 16-detector row helical CT. This scanner enables simultaneous acquisition of up to 45 transaxial PET images with inter-slice spacing of 4 mm in one bed position, and provides an image from the vertex to the thigh with about 10 bed positions. The transaxial field of view and pixel size of the PET images reconstructed for fusion were 57.6 cm and 4 mm, respectively, with a matrix size of 144×144. The technical parameters used for CT imaging were as follows: pitch 0.8, gantry rotation speed 0.5 s/rotation, 120 kVp, 250 mAs, 3 mm slice thickness, and specific breath-holding instructions.

After 4-6 h of fasting, patients received an intravenous injection of 370 MBq F-18 FDG. About 75 min later, CT images were obtained from the vertex to the mid-thigh for about 32 s. In cases with which intravenous contrast material was used, CT scans were obtained 60 s after injection of 2 mL/kg of non-ionic contrast material (Omnipaque 300; GE Healthcare). An emission PET scan followed in 3D acquisition mode for the same longitudinal coverage, 1.5 min per bed position. CT images were fused with the PET data and were used to generate a map for attenuation correction. PET images were reconstructed using a line of response protocol with CT attenuation correction and the reconstructed images were generated for review on a computer workstation (Extended Brilliance Workstation, Philips Medical Systems, Cleveland OH, USA).

The data processing resulted in building a solid matching matrix between 3D regions before treatment to their corresponding 3D regions after treatment. The verification of the result was done in collaboration with a doctor, who authenticated the matching results with the existing raw regions. The results were classified by statistical measures that include: True Positive (TP)—regions that were detected to be connected by the system and found to be true; True Negative (TN)—Regions that were detected by the system as non-connected and found to be unconnected; False Positive (FP)—Regions that were found to be connected by the system while they were not; and False Negative (FN)—Regions that were found to be unconnected by the system, while they were.

All of the above was summarized to two main statistical measures, Sensitivity and Specificity, given by:

$$Sensativity = \frac{T P}{T P + F N} \qquad ( 4 a )$$

$$Specificity = \frac{T N}{T N + F P} \qquad ( 4 b )$$

The results obtained by the inventors provided total Sensitivity of 91.96 and Specificity of 99.56.

Also, the system of the invention provided the ability to estimate changes between matching regions in one or more parameters, such as their volume, activity level (SUV) and roughly HU levels.

Thus, the inventors have shown that the system can serve as a helping tool to doctors when examining the difference between two different sets of PET-CT scans for a given patient.

Thus, the present invention provides a novel and unique approach to a fully automated system for detecting correlation and non-correlation between suspicious pathological regions in PET-CT scans before and after treatment.

The PET-CT gives a robust approach for diagnosis. The aim of the PET scan is to provide a 3D picture of the whole human body and the distribution of the isotope through it. The inventors have shown that by choosing a certain level of threshold (defined by a physician), which deliberately was very low, the suspicious regions can be easily separated and data from them can be extracted. It should be noted that the technique of the invention is not aimed at determining a possibility of a tumour development, but at presenting differences between suspicious regions, which worth a further observation for a given SUV level of interest by the physician.

It should be noted, although not specifically illustrated, the technique of the present invention (the above-described algorithm) provides for a fully automated user interface presenting to a physician (radiologist) the changes of such parameters as HU, SUV and Volume of all lesions and within each specific lesion. The interface can show the following data: raw PET data including full body scan slices of 2D Coronal images PET-FBCI and 2D Sagittal images PET-FBSI of the post-treatment stage and possibly also of the pre-treatment stage (which have been previously duly recorded and stored in the system); corresponding CT data including 2D Coronal image CT-FBCI and 2D Sagittal image CT-FBSI; fusion data, i.e. CT-PET Coronal and CT-PET Sagittal images CT-PET-CI and CT-PET-SI, as well as various cross-sectional views. Also presented in the interface is a body part 3D image showing automatically identified and selected lesions; and heat map data for each and any of these lesions, presenting clear measures for the changes in all the relevant parameters. The interface enables radiologist to select the lesion whose heat map is to be displayed, as well as locations through the specific lesion.

Thus, the system of the invention can receive a full PET-CT scan as an input and produce an output of full-body 3D, and 2D images of the PET-CT scan with all the suspicious findings marked automatically. This could help the physician focus on the image's important findings with distractions of less important findings. Also, the system can receive a full PET-CT scan as input and produce an output of full-body 3D and 2D images of the PET-CT scan with a “heat map” that presents the changes from the previous scan. Hence, there will be no need to manually choose a specific finding/lesion in order to examine its difference from the previous scan. This difference includes parameters such as SUV, HU, volume, heterogeneity, mean level, peak lesion level, etc. Such user interface can help the physician to analyse the scan and write the scan interpretation more quickly. Moreover, a possible output of the system might be an automated report of all the findings in the scans and their difference from the previous test. This report could, for instance, be recorded in the patient medical documentation or be used for research.

Thus, the inventors have developed a novel fully automatic technique that provides for detecting changes in size of suspicious regions, SUV & HU levels from two sets of data (Before and After treatment). Such fully automatic detection of these changes, simplifies and assists radiologists and nuclear medicine physicians in their work during medical diagnosis.

1. A system for monitoring treatment of a patient, the system comprising data input and output utilities, memory, and a data processor, and being configured for data communication with an image data provider to receive from the image data provider image data indicative of combined PET-CT scan images including at least one first pre-treatment full body scan image of a patient and at least one second post-treatment full body scan image of the patient, wherein said data processor comprises: an identifier utility configured to process the image data to identify matching first and second 2D regions in, respectively, first 2D slices forming the first pre-treatment full body scan image and second 2D slices forming the second post-treatment full body scan image, and locate at least one pair of corresponding first and second 3D regions in the first and second full body scan images, respectively; and an analyzer configured and operable to analyze each of said at least one pair of the first and second 3D regions and determine a change in at least one parameter of interest in the first and second 3D regions, and generate output data indicative of said change.

2. The system according to claim 1, wherein said data processor is configured to represent each slice in a first set of the 2D slices forming the first full body scan image and each slice in a second set of the 2D slices forming the second full body scan image by respective first 2D logical image and second 2D logical image, the first and second 2D logical images comprising first Coronal and first Sagittal pre-treatment images, and second Coronal and second Sagittal post-treatment images.

3. The system according to claim 2, wherein said data processor comprises:
a data transformation utility configured to transform 3D data indicative of each of said at least one first full body scan image and each of said at least one second full body scan image into respective first and second pairs of 2D logical images, the 2D logical images of the first pair corresponding to a first pre-treatment Coronal image and a first pre-treatment Sagittal image of the patient's body and the 2D logical images of the second pair corresponding to a second post-treatment Coronal image and a second post-treatment Sagittal image of the patient's body;
a match finding utility configured to process data indicative of the first and second pairs of the 2D logical images and define one or more pairs of matching 2D regions in said first and second pairs of the 2D logical images, each pair of the matching 2D regions being defined by first 2D regions in the first Coronal and Sagittal images matching with respective second 2D regions in the second Coronal and Sagittal images; and
a region defining utility configured to analyse each of said one or more pairs of the matching 2D regions over the first and second full-body scan images, and define and locate a corresponding pair of matching first and second 3D regions in the first and second full body scan images, such that the first 3D region in the first full body scan image corresponds to the first 2D regions in the first Coronal and first Sagittal images, and the second 3D region in the second full body scan image corresponds to the second 2D regions in the second Coronal and Sagittal images, thereby determining data about said one or more 3D regions to be analysed to identify changes between pre-treatment and post-treatment.

4. The system according to claim 3, wherein said region defining utility has at least one of the following configurations:
region defining utility is configured to determine a solid matching matrix between the one or more 3D regions pre-treatment to corresponding one or more 3D regions post-treatment, thereby enabling to identify the changes in one or more parameters of the matching regions;
the region defining utility is configured to determine a solid matching matrix between the one or more 3D regions pre-treatment to corresponding one or more 3D regions post-treatment, thereby enabling to identify data indicative of one or more new regions that emerged and data indicative of old regions that disappeared, as a result of said treatment.

5. (canceled)

6. The system according to claim 1, wherein said one or more parameters comprise at least one of the following: volume, activity level (SUV) and CT scale of density (HU) levels.

7. The system according to claim 3, wherein said data processor comprises a pre-processor configured to pre-process raw data indicative of said first and second full body scan images to define said 3D data to be processed by the data transformation utility, said pre-processor comprising:
a normalization utility configured to normalize data corresponding to each of the first and second full body scan images to a predetermined scale thereby obtaining respective first and second normalized full body scan images; and
a filtering utility configured to apply spatial and thresholding filtering to each of said first and second full body normalized scan images and provide corresponding first and second sets of filtered 2D PET slices forming said first and second full body scan images, respectively.

8. The system according to claim 7, wherein said predetermined scale corresponds to Standard Uptake Value (SUV).

9. The system according to claim 8, wherein said data processor is configured to utilize input data indicative of weight of the patient, active isotope dose used while obtaining said scan image data, and scan time, in order to perform the normalization of the scan image data to the SUV scale.

10. The system according to claim 7, wherein the data transformation utility is further configured and operable to apply a dimension reduction procedure to the filtered 2D PET-CT slices to optimize the 2D logical images.

11. The system according to claim 3, wherein said data transformation utility is configured to perform registration of each of the 2D logical images of the first pair in X- and Y axes to correspond to shifts of the patient's body between scans during imaging.

12. The system according to claim 11, wherein said data transformation utility is further configured to perform segmentation of each of the 2D logical images to define each individual region in the 2D logical image, and determine parameters of each of the individual regions.

13. The system according to claim 12, wherein the data transformation utility is configured for matching at least some of said parameters of each individual region of the first 2D logical image before registration with parameters of the region in the first 2D logical image after registration.

14. The system according to claim 3, wherein the match finding utility is configured to represent each of the 2D regions in 3D space and in 2D space by performing 2D Cross-Correlation, to determine a distance, R, by which the first pre-treatment image is to be moved for matching the second post-treatment image, for each of the Coronal and Sagittal 2D images.

15. The system according to claim 14, wherein the match finding utility is configured to carry out the following:
apply first Cross-Correlations to each 2D region of the first pre-treatment Coronal 2D image with respect to all 2D regions in the second post-treatment Coronal image, and to each 2D region of the first pre-treatment Sagittal 2D image with respect to all 2D regions in the second post-treatment Sagittal image, thereby obtaining a first Coronal matrix, CZ^{(BT)}×CZ^{(AT)}, with a size CZ^{(BT)} of pre-treatment Coronal regions on a size CZ^{(AT)} of post-treatment Coronal regions, and a first Sagittal matrix, SZ^{(BT)}×SZ^{(AT)} with a size SZ^{(BT)} of pre-treatment Sagittal regions on a size SZ^{(AT)} of post-treatment Sagittal regions, each cell in the matrix representing the distance R;
apply a second Cross-Correlation in determined limits to the regions of pre-treatment and post-treatment Coronal and Sagittal images regions and obtaining, for each of the Coronal and Sagittal images, a first matrix of a size of number of pre-treatment regions on a size of post-treatment regions, and a second matrix of the size of number of after-treatment regions on the number of pre-treatment regions, content of the first and matrixes being the distance R;
thereby obtaining six matrixes comprised of three matrixes for the Coronal image and three matrixes for the Sagittal image.

16. The system according to claim 15, wherein the match finding utility is configured to optimize a matching process by carrying out the following: applying revoking to each of said six matrixes to revoke one or more illogical matches, being a match of the region in the pre-treatment image to several regions in a corresponding post-treatment image; and merging resulting six matrixes into 2D Coronal-Merge matrix and 2D Sagittal-Merge matrix.

17. The system according to claim 16, wherein the match finding utility is configured to verify validity of the Coronal-Merge and Sagittal-Merge matrixes to regions of the post-treatment Coronal and Sagittal images that have not passed through the registration, and thereby obtain two 2D matrixes including relations between the regions without registration, for the pre-treatment and post-treatment Coronal and Sagittal images.

18. A method for monitoring treatment of a patient, the method comprising:
providing image data indicative of combined PET-CT scan images including at least one first pre-treatment full body scan image of a patient and at least one second post-treatment full body scan image of the patient,
processing the image data by carrying out the following: identifying matching first and second 2D regions in, respectively, first 2D slices forming the first pre-treatment full body scan image and second 2D slices forming the second post-treatment full body scan image; locating at least one pair of corresponding first and second 3D regions in the first and second full body scan images, respectively, and analysing each of said at least one pair of the first and second 3D regions to determine a change in at least one parameter of interest in the first and second 3D regions, and generating output data comprising data indicative of said change.

19. The method according to claim 18, wherein said output data comprises data indicative of one or more new regions that emerged and data indicative of old regions that disappeared.

20. The method according to claim 18, wherein said processing comprises representing each slice in a first set of 2D slices forming the first full body scan image and each slice in a second set of 2D slices forming the second full body scan image by respective first 2D logical image and second 2D logical image, the first and second 2D logical images comprising first Coronal and first Sagittal pre-treatment images, and second Coronal and second Sagittal post-treatment images.

21. The method according to claim 20, wherein said processing comprises:
transforming 3D data indicative of each of said at least one first full body scan image and each of said at least one second full body scan image into respective first and second pairs of 2D logical images, the 2D logical images of the first pair corresponding to a first pre-treatment Coronal image and a first pre-treatment Sagittal image of the patient's body and the 2D logical images of the second pair corresponding to a second post-treatment Coronal image and a second post-treatment Sagittal image of the patient's body;
processing data indicative of the first and second pairs of the 2D logical images and defining one or more pairs of matching 2D regions in said first and second pairs of the 2D logical images, each pair of the matching 2D regions being defined by first 2D regions in the first Coronal and Sagittal images matching with respective second 2D regions in the second Coronal and Sagittal images; and
analysing each of said one or more pairs of the matching 2D regions over the first and second full-body scan images, and defining and locating a corresponding pair of matching first and second 3D regions in the first and second full body scan images, such that the first 3D region in the first full body scan image corresponds to the first 2D regions in the first Coronal and first Sagittal images, and the second 3D region in the second full body scan image corresponds to the second 2D regions in the second Coronal and Sagittal images, thereby determining data about said one or more 3D regions to be analysed to identify changes between pre-treatment and post-treatment.

22. The method according to claim 21, wherein said analyzing comprises determining a solid matching matrix between the one or more pre-treatment 3D regions and corresponding one or more post-treatment 3D regions post-treatment, thereby enabling to identify the changes in one or more parameters of the matching regions.

23. The method according to claim 18, wherein said one or more parameters comprise at least one of the following: volume, activity level (SUV) and CT scale of density (HU) levels.

24. The method according to claim 20, further comprising pre-processing raw data indicative of said first and second full body scan images to define said 3D data for the transformation, said pre-processing comprising:
normalizing data corresponding to each of the first and second full body scan images to a predetermined scale thereby obtaining respective first and second normalized full body scan images; and
applying spatial and thresholding filtering to each of said first and second full body normalized scan images and providing corresponding first and second sets of filtered 2D PET slices forming said first and second full body scan images, respectively.

25. The method according to claim 24, wherein said predetermined scale corresponds to Standard Uptake Value (SUV).

26. The method according to claim 25, wherein said pre-processing utilizing input data indicative of weight of the patient, active isotope dose used while obtaining said scan image data, and scan time, in order to perform the normalization of the scan image data to the SUV scale.

27. The method according to claim 21, wherein said transforming further comprises: applying a dimension reduction procedure to the filtered 2D PET-CT slices to optimize the 2D logical images.

28. The method according to claim 21, wherein said transforming further comprises: performing registration of each of the 2D logical images of the first pair in X- and Y axes to correspond to shifts of the patient's body between scans during imaging.

29. The method according to claim 28, wherein said transforming comprises: performing segmentation of each of the 2D logical images to define each individual region in the 2D logical image, and determining parameters of each of the individual regions.

30. The method according to claim 29, wherein said transforming comprises: matching at least some of said parameters of each individual region of the first 2D logical image before registration with parameters of the region in the first 2D logical image after registration.

31. The method according to claim 21, wherein said matching comprises: representing each of the 2D regions in 3D space and in 2D space by performing 2D Cross-Correlation, to determine a distance, R, by which the first pre-treatment image is to be moved for matching the second post-treatment image, for each of the Coronal and Sagittal 2D images.

32. The method according to claim 31, wherein said matching comprises:
applying first Cross-Correlations to each 2D region of the first pre-treatment Coronal 2D image with respect to all 2D regions in the second post-treatment Coronal image, and to each 2D region of the first pre-treatment Sagittal 2D image with respect to all 2D regions in the second post-treatment Sagittal image, thereby obtaining a first Coronal matrix, CZ^{(BT)}×CZ^{(AT)}, with a size CZ^{(BT)} of pre-treatment Coronal regions on a size CZ^{(AT)} of post-treatment Coronal regions, and a first Sagittal matrix, SZ^{(BT)}×SZ^{(AT)} with a size SZ^{(BT)} of pre-treatment Sagittal regions on a size SZ^{(AT)} of post-treatment Sagittal regions, each cell in the matrix representing the distance R;
applying a second Cross-Correlation in determined limits to the regions of pre-treatment and post-treatment Coronal and Sagittal images regions and obtaining, for each of the Coronal and Sagittal images, a first matrix of a size of number of pre-treatment regions on a size of post-treatment regions, and a second matrix of the size of number of after-treatment regions on the number of pre-treatment regions, content of the first and matrixes being the distance R;
thereby obtaining six matrixes comprised of three matrixes for the Coronal image and three matrixes for the Sagittal image.

33. The method according to claim 32, wherein said matching comprises: applying revoking to each of said six matrixes to revoke one or more illogical matches, being a match of the region in the pre-treatment image to several regions in a corresponding post-treatment image; and merging resulting six matrixes into 2D Coronal-Merge matrix and 2D Sagittal-Merge matrix.

34. The method according to claim 33, wherein the matching comprises: verifying validity of the Coronal-Merge and Sagittal-Merge matrixes to regions of the post-treatment Coronal and Sagittal images that have not passed through the registration, and thereby obtaining two 2D matrixes including relations between the regions without registration, for the pre-treatment and post-treatment Coronal and Sagittal images.

35. The method according to claim 18, comprising: receiving the combined PET-CT scan image and generating and presenting on a user interface corresponding full-body 3D image and 2D images of the PET-CT scan in which all the suspicious regions are automatically marked.

36. The method according to claim 18, comprising receiving the combined full body pre-treatment and post-treatment PET-CT scans and generating and presenting on user interface corresponding full-body 3D and 2D images of the post-treatment PET-CT scan with a heat map presenting changes from the pre-treatment scan in one or more of the following parameters: SUV, HU, volume, heterogeneity, mean level, peak lesion level.

37. The method according to claim 18, further comprising automatically generating a report of all findings in the post-treatment scans and their difference from the pre-treatment.
